(19) United States
(12) Patent Application Publication
(10) Pub. No.: US 2021/0008229 A1
(43) Pub. Date: Jan. 14, 2021

(54) TARGETED PHOTOACOUSTIC AGENTS AND USES THEREOF

(71) Applicant: McMaster University, Hamilton (CA)

(72) Inventors: John Valliant, Ancaster (CA); Samantha Slikboer, Seagrave (CA); Zoya Naperstkow, Oakville (CA)

(21) Appl. No.: 16/926,807

(22) Filed: Jul. 13, 2020

Related U.S. Application Data
(60) Provisional application No. 62/873,237, filed on Jul. 12, 2019.

(30) Foreign Application Priority Data
Jul. 12, 2019 (CA) 3049352

(51) Int. Cl. A61K 49/22; C09B 23/01

(52) U.S. Cl. CPC A61K 49/221; C09B 23/0066

(57) ABSTRACT

The present application relates to photoacoustic agents, to kits comprising such photoacoustic agents and to uses thereof such as in methods for detecting a presence of a target in a subject. The photoacoustic agents comprise a photoacoustically active moiety (for example, a near infrared dye) that is coupled to a first bioorthogonal reactive group and optionally a targeting entity for a target that is coupled to a second bioorthogonal reactive group.

### RELATED APPLICATIONS

The present application claims the benefit of priority of co-pending U.S. provisional patent application No. 62/873,237 filed on Jul. 12, 2019, and Canadian patent application no. 3,049,352 filed on Jul. 12, 2019, the contents of each of which are incorporated herein by reference in their entirety.

### FIELD

The present application relates to photoacoustic agents and to uses thereof, in particular in methods for targeted photoacoustic imaging, and to kits comprising such photoacoustic agents.

### BACKGROUND

Photoacoustic imaging is an emerging modality that assuages the limited depth of penetration associated with traditional optical methods. The technique involves the generation of acoustic waves following irradiation of suitable contrast agents with rapid laser pulses. These pulses cause endogenous tissue chromophores or exogeneous contrast agents to rapidly expand and contract, producing acoustic waves at megahertz frequencies.¹ Photoacoustic imaging can penetrate several centimeters while having about 100 μm resolution, and may be used, for example, preclinically to evaluate tumor models and test new therapies,^{2,3} and clinically as an experimental alternative to methods that utilize ionizing radiation for imaging breast, and head and neck cancers.⁴

There have been extensive efforts to develop photoacoustic imaging agents derived from nanomaterials and low molecular weight dyes.^{4,5,6,7,8} One active area of research is to create contrast agents that are capable of targeting specific cancer biomarkers.^{9,10,11} To this end, low molecular weight photoacoustic imaging dyes have been linked to cancer cell-seeking small molecules and antibodies to concentrate the contrast agent selectively within tumors.^{10,12,13} Work to date has largely been accomplished using indocyanine green (ICG) and conventional ligation chemistry where there is a limited toolbox of general-purpose strategies available for creating targeted photoacoustic imaging probes that can be employed by non-synthetic chemistry experts.

### SUMMARY

The complexities involved in preparing molecularly targeted photoacoustic imaging probes has hindered the ability of the broader scientific community to employ and fully exploit the unique features of photoacoustic imaging. To address this, a new general-purpose photoacoustic dye was synthesized that is an easy to use platform for creating targeted imaging agents in a single step using biorthogonal chemistry. Following an efficient two-step synthesis from an inexpensive commercially available dye, as an exemplar, the platform was used to create a photoacoustic probe for imaging calcium accretion where high resolution photoacoustic images of the knee joint in mice were obtained as early as one hour post-injection. Whole-body distribution was determined subsequently by labeling with ^{99m}Tc and performing tissue counting following necropsy. These studies, along with tumour imaging and in vitro binding studies, revealed that the core photoacoustic agent is also an albumin binding ligand and has a useful biological half-life and distribution profile to go along with the straightforward way it can be linked to targeting molecules.

Accordingly, the present application includes a photoacoustic agent comprising a photoacoustically active moiety coupled to a first bioorthogonal reactive group. In an embodiment, the photoacoustically active moiety is a near infrared (NIR) dye. In another embodiment, the photoacoustic agent further comprises a targeting entity for a target. In a further embodiment, the targeting entity is coupled to a second bioorthogonal reactive group that is complementary to the first bioorthogonal reactive group and the first bioorthogonal reactive group and the second bioorthogonal reactive group form a bioorthogonal complex.

The present application also includes a method for detecting a presence of a target in a subject, comprising:
administering a photoacoustic agent to the subject, the photoacoustic agent comprising a photoacoustically active moiety coupled to a first bioorthogonal reactive group and a targeting entity for the target;
allowing the targeting entity to localize to the target; and
imaging the subject using photoacoustic imaging to detect the photoacoustic agent, wherein the detection of the photoacoustic agent indicates the presence of the target in the subject.

The present application also includes a method for detecting a presence of a target in a subject, comprising:
administering a targeting agent to the subject, the targeting agent comprising a targeting entity for the target coupled to a second bioorthogonal reactive group;
allowing the targeting entity to localize to the target;
administering a photoacoustic agent to the subject, the photoacoustic agent comprising a photoacoustically active moiety coupled to a first bioorthogonal reactive group that is complementary to the second bioorthogonal reactive group such that the first bioorthogonal reactive group and the second bioorthogonal reactive group form a bioorthogonal complex; and
imaging the subject using photoacoustic imaging to detect the photoacoustic agent, wherein the detection of the photoacoustic agent indicates the presence of the target in the subject.

The present application also includes a kit comprising:
a photoacoustic agent, the photoacoustic agent comprising a photoacoustically active moiety coupled to a first bioorthogonal reactive group;
a targeting agent, the targeting agent comprising a targeting entity for a target coupled to a second bioorthogonal reactive group that is complementary to the first bioorthogonal reactive group such that the first bioorthogonal reactive group and the second bioorthogonal reactive group form a bioorthogonal complex; and
optionally instructions for use of the targeting agent and the photoacoustic agent in imaging a subject using photoacoustic imaging to detect the photoacoustic agent.

Other features and advantages of the present application will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

### BRIEF DESCRIPTION OF THE DRAWINGS

The present application will now be described in greater detail with reference to the drawings in which:

FIG. 1 shows high performance liquid chromatography (HPLC) traces showing the plasma stability of a dye-tetrazine conjugate according to an embodiment of the present application before addition to plasma (top trace), 24 h after incubating at 37° C. in plasma (middle trace), and reacting with a trans-cyclooctene-labelled bisphosphonate (TCO-BP) after 24 h in plasma (bottom trace).

FIG. 2 shows a photoacoustic (PA average) threshold spectrum (upper spectrum) and an absorbance spectrum (lower spectrum) of a dye-tetrazine conjugate according to an embodiment of the present application.

FIG. 3 shows photoacoustic spectra of 0.1 mM of a TCO-conjugate of a dye-tetrazine conjugate according to an embodiment of the present application in saline (black) and blood (grey).

FIG. 4 shows B-Mode and photoacoustic images of 0.1 mM of a TCO-conjugate of a dye-tetrazine conjugate according to an embodiment of the present application in blood (left), and saline (right).

FIG. 5 shows normalized PA spectra of 0.1 mM of a dye-tetrazine conjugate according to an embodiment of the present application in saline (lower trace) and blood (upper trace) (upper plot) and PA average threshold vs. concentration of a dye-tetrazine conjugate according to an embodiment of the present application at maxima in phantom (775 nm) (lower plot).

FIG. 6 shows overlay B-Mode and photoacoustic image in phantom with spectral unmixing of 0.1 mM of a dye-tetrazine conjugate according to an embodiment of the present application in blood, and 0.1 mM of the dye-tetrazine conjugate according to an embodiment of the present application in saline.

FIG. 7 shows photoacoustic images of cotton swabs dipped in increasing concentrations of a dye-tetrazine conjugate according to an embodiment of the present application (1 mM (top), 2 mM (middle), and 4 mM (bottom)) (upper images). In color images red is most intense signal, blue is least intense signal. FIG. 7 also shows a plot of the average photoacoustic signal at 714 nm vs concentration for the cotton swabs imaged in upper images (lower plot).

FIG. 8 shows photoacoustic images of a 143B human osteosarcoma mouse xenograft tumor prior to administration (upper left) and 1 hour after administration of a dye-tetrazine conjugate according to an embodiment of the present application (upper right) as well as a photoacoustic image of the same tumor shown in the upper right image ex vivo 1 hour after injection (lower left) and a graph of the average signal intensity in the tumor before and after administration of the dye-tetrazine conjugate according to an embodiment of the present application showing a 2.3× increase over background (lower right). Red in color images=oxyhemoglobin, Blue in color images=deoxyhemoglobin and Green in color images=the dye-tetrazine conjugate according to an embodiment of the present application. The dashed white lines outline the tumor.

FIG. 9 shows photoacoustic images of a mouse leg focusing on the knee joint at 1 hour after injection of a dye-tetrazine conjugate according to an embodiment of the present application (upper left), the same mouse leg ex vivo (upper right), a mouse leg 1 hour after injection of a TCO-conjugate of a dye-tetrazine conjugate according to an embodiment of the present application (lower left) and a photoacoustic image of the same mouse leg ex vivo (lower right). The white arrows indicate the knee joint and light grey arrows indicate blood vessels.

FIG. 10 shows radioTLC traces of TCO-BP labeled with ^{99m}Tc; Upper trace: Acetonitrile radioTLC showing 2.66% free pertechnetate; and Lower trace: Water radioTLC showing 11% colloidal ^{99m}Tc.

FIG. 11 is a plot showing biodistribution data for selected fluids and tissues for TCO-BP labeled with ^{99m}Tc and coupled to a dye-tetrazine conjugate according to an embodiment of the present application. Experiments were performed using Balb/c mice (n=3 per time point) and tissues and fluids were collected at 1, 3, 6, and 24 h post administration. Data are expressed as the mean percent injected dose per gram (% ID g−1)±SEM.

FIG. 12 shows a plot of blood concentrations expressed as percent injected dose per gram (% ID g−1) for TCO-BP labeled with ^{99m}Tc (black) and TCO-BP labeled with ^{99m}Tc and coupled to a dye-tetrazine conjugate according to an embodiment of the present application (grey) at 1 and 6 h p.i. (upper plot) and fluorescence spectra and intensity data for blood samples (n=3 for each time point) following the administration of TCO-BP labeled with ^{99m}Tc and coupled to a dye-tetrazine conjugate according to an embodiment of the present application taken at 1, 3, 6, and 24 h p.i. (lower plot).

FIG. 13 is a plot showing the results of a plasma binding study of TCO-BP labeled with ^{99m}Tc and coupled to a dye-tetrazine conjugate according to an embodiment of the present application.

FIG. 14 shows absorption spectra of a dye-tetrazine conjugate according to an embodiment of the present application (3 μM) with increasing concentrations of bovine serum albumin (BSA) (top left); absorbance at 776 nm versus concentration of BSA (top left); fluorescence intensity of the dye-tetrazine conjugate according to an embodiment of the present application (3 μM) with increasing concentrations of BSA (λₑₓ=736 nm) (bottom left; and fluorescence intensity at 786 nm versus concentration of BSA (bottom right).

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “and/or” as used herein means that the listed items are present, or used, individually or in combination. In effect, this term means that “at least one of” or “one or more” of the listed items is used or present.

As used in this application, the singular forms “a”, “an” and “the” include plural references unless the content clearly dictates otherwise.

The term “suitable” as used herein means that the selection of specific reagents or conditions will depend on the reaction being performed and the desired results, but none-the-less, can generally be made by a person skilled in the art once all relevant information is known.

The term “subject” as used herein includes all members of the animal kingdom including mammals, and suitably refers to humans.

The term “countercation” as used herein refers to a positively charged species consisting of a single element, or a positively charged species consisting of a group of elements connected by ionic and/or covalent bonds.

The term “pharmaceutically acceptable” means compatible with use in subjects, for example, mammals such as humans.

The term “bioorthogonal reactive group” as used herein refers to a moiety capable of undergoing a reaction with a complementary bioorthogonal reactive group in vivo without interfering with native biochemical processes.

The term “bioorthogonal complex” as used herein refers to a compound comprising a covalent linkage formed by the reaction of a first bioorthogonal reactive group with a second bioorthogonal reactive group that is complementary to the first bioorthogonal reactive group.

The term “photoacoustically active moiety” as used herein refers to a chemical moiety that generates a signal based on the absorption of laser-generated optical energy leading to acoustic emissions that are detectable by an ultrasound transducer. In a typical system, upon irradiation with a nonionizing short-pulsed laser beam, the photoacoustically active moiety absorbs the optical energy, undergoes thermoelastic expansion and produces photoacoustic waves that are received with a wide-band ultrasonic transducer (e.g. 5-50 MHz).

The term “coupled” as used herein refers to any suitable means for chemically linking two molecular structures together. For example, in some embodiments, the two molecular structures are linked directly via a bond. In other embodiments, the two molecular structures are linked together via a linker group.

The term “linker group” as used herein refers to any suitable molecular structure that joins two molecular structures together.

The term “aryl” as used herein refers to cyclic groups that contain at least one aromatic ring. In an embodiment of the present application, the aryl group contains from 6, 9, 10 or 14 atoms, such as phenyl, naphthyl, indanyl or anthracenyl. The number of carbon atoms that are possible in the referenced aryl group are indicated by the numerical prefix “Cₙ₁₋ₙ₂”. For example, the term C₆₋₁₀aryl means an aryl group having 6, 7, 8, 9 or 10 carbon atoms.

### II. Photoacoustic Agents

A new general-purpose photoacoustic dye was synthesized that is an easy to use platform for creating targeted imaging agents in a single step using biorthogonal chemistry. Following an efficient two-step synthesis from an inexpensive commercially available dye, as an exemplar, the platform was used to create a photoacoustic probe for imaging calcium accretion where high resolution photoacoustic images of the knee joint in mice were obtained as early as one hour post-injection. Whole-body distribution was determined subsequently by labeling with ^{99m}Tc and performing tissue counting following necropsy. These studies, along with tumour imaging and in vitro binding studies, revealed that the core photoacoustic agent is also an albumin binding ligand and has a useful biological half-life and distribution profile to go along with the straightforward way it can be linked to targeting molecules. The new photoacoustic dye creates, for example, the opportunity to employ the selectivity and versatility of inverse electron demand Diels-Alder (IEEDA) chemistry between tetrazines and trans-cyclooctene (TCO) to create new photoacoustic contrast agents.

Accordingly, the present application includes a photoacoustic agent comprising a photoacoustically active moiety coupled to a first bioorthogonal reactive group.

The photoacoustically active moiety can be any suitable photoacoustically active moiety. In an embodiment, the photoacoustically active moiety is selected from a gold nanoparticle, a carbon nanomaterial, a polymer nanoparticle, a genetically encoded chromophore and a near infrared (NIR) dye.

In an embodiment, the photoacoustically active moiety is a gold nanoparticle. The gold nanoparticle can be any suitable gold nanoparticle. For example, it will be appreciated by a person skilled in the art that gold nanoparticles can be tuned to exhibit absorption peaks in the NIR region. In an embodiment, the gold nanoparticle is selected from a nanorod, nanosphere, nanocage, nanoshell, nanocluster, nanobeacon, nanostar and combinations thereof.

In an embodiment, the photoacoustically active moiety is a carbon nanomaterial. The carbon nanomaterial can be any suitable carbon nanomaterial. In another embodiment, the carbon nanomaterial is a carbon nanotube, carbon nanorod, or carbon nanosheet.

In an embodiment, the photoacoustically active moiety is a polymer nanoparticle. The polymer nanoparticle can be any suitable polymer nanoparticle. In an embodiment, the polymer nanoparticle is a semiconducting polymer nanoparticle. Exemplary semiconducting polymer nanoparticles may be found, for example, in L. Cui and J. Rao, “Semiconducting polymer nanoparticles as photoacoustic molecular imaging probes” Wiley Interdiscip Rev Nanomed Nanobiotechnol. 2017 March; 9(2)).

In an embodiment, the photoacoustically active moiety is a genetically encoded chromophore. The genetically encoded chromophore can be any suitable genetically encoded chromophore. Exemplary genetically encoded chromophores may be found, for example, in J. Brunker et al., “Photoacoustic imaging using genetically encoded reporters: a review” J. Biomed. Opt. 2017, 22(7), 070901.

In an embodiment, the photoacoustically active moiety is a near infrared (NIR) dye. The NIR dye can be any suitable NIR dye. The dye used in the examples of the present application was found to bind to albumin. The ability to bind to blood proteins such as albumin may be useful, for example, to prolong circulation of the photoacoustic agent. Accordingly, in another embodiment, the NIR dye is capable of binding a blood protein. In an embodiment, the blood protein is albumin.

In an embodiment, the NIR dye is a sulfonated cyanine dye.

In an embodiment, the NIR dye is of Formula (I):

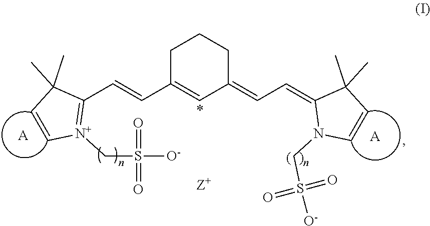

wherein

each

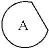

is independently aryl;

each n is independently an integer of from 2 to 8; Z⁺ is a pharmaceutically acceptable countercation; and

* is the site at which the NIR dye is coupled to the first bioorthogonal reactive group.

In an embodiment, each

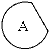

is independently C₆₋₁₀aryl. In another embodiment each is

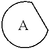

independently phenyl or naphthyl. In a further embodiment, both

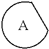

are phenyl. In another embodiment, both

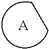

are naphthyl.

In an embodiment, each n is independently an integer of from 2 to 6. In another embodiment, each n is independently an integer of from 3 to 5. In a further embodiment of the present application both n are 4.

In an embodiment, the NIR dye is of the formula:

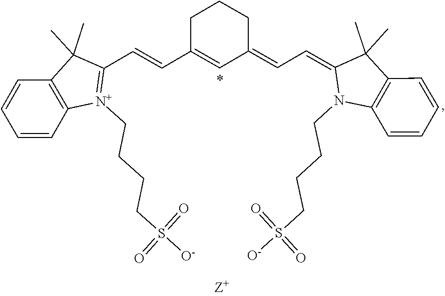

wherein

Z⁺ is a pharmaceutically acceptable countercation; and

* is the site at which the NIR dye is coupled to the first bioorthogonal reactive group.

Z⁺ is any suitable pharmaceutically acceptable countercation. In an embodiment of the present application, Z⁺ is Na⁺.

In an embodiment, both

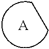

are phenyl; both n are 4; and Z⁺ is Na⁺. In another embodiment, both

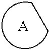

are naphthyl; both n are 4; and Z⁺ is Na⁺.

In an embodiment, the photoacoustically active moiety is coupled to the first bioorthogonal reactive group by a linker group. The linker group can be any suitable linker group. In an embodiment, the linker group is obtained by a method comprising Suzuki-Miyaura coupling between a photoacoustically active moiety having a halide (e.g. CI) at the site designated * and a suitable diboronic acid having an aldehyde moiety in the presence of Pd(PPh₃)₄ and Cs₂CO₃, then reacting the aldehyde thereby obtained with a first bioorthogonal reactive group having a hydrazyl group under suitable conditions (e.g. conditions described in Vito et al., PLoS One 2016, 11 e0167425) to obtain the corresponding hydrazone. For example, in an embodiment, the linker group is of the formula:

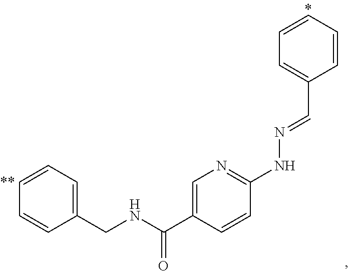 [SMILES: O=C(NCc1ccccc1)c1ccc(N/N=C/c2ccccc2)nc1]

wherein

* is the site that is bonded to the photoacoustically active dye; and

** is the site that is bonded to the first bioorthogonal reactive group.

In an another embodiment of the present application, the linker group is a suitable polyethylene glycol (PEG).

In an embodiment, the photoacoustic agent further comprises a targeting entity for a target. In another embodiment, the targeting entity is an antibody, a receptor ligand or a bisphosphonate. In a further embodiment, the target is a cellular marker for one of inflammation, cancer, a skeletal condition, a heart abnormality, atherosclerosis, angiogenesis and intravascular thrombus formation. In an embodiment, the skeletal condition is microfractures.

In an embodiment, the targeting entity is coupled to a second bioorthogonal reactive group that is complementary to the first bioorthogonal reactive group and the first bioorthogonal reactive group and the second bioorthogonal reactive group form a bioorthogonal complex. In another embodiment, the first bioorthogonal reactive group and the second bioorthogonal reactive group, in either order, are inverse electron demand Diels Alder reaction pairs, or an azide and a functionalized phosphine, or an azide and a strained alkyne. In a further embodiment, the first bioorthogonal reactive group and the second bioorthogonal reactive group, in either order, are tetrazine and trans cyclooctene. In another embodiment, the first bioorthogonal reactive group is tetrazine and the second bioorthogonal reactive group is trans cyclooctene.

In an embodiment, the photoacoustic agent is of the formula:

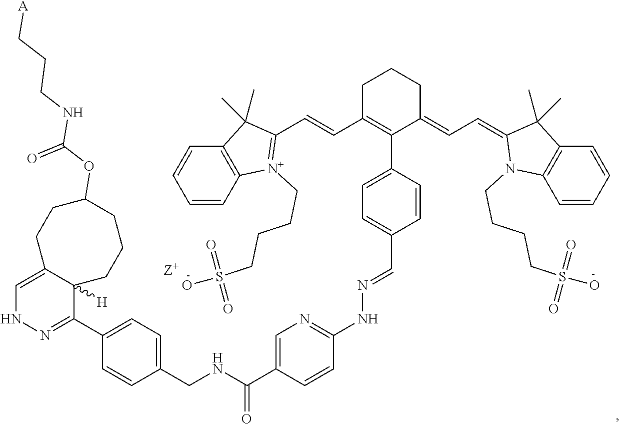

wherein

Z⁺ is a pharmaceutically acceptable countercation as defined herein; and

A is the targeting entity.

### III. Methods and Kits

A photoacoustic probe was synthesized and used for imaging calcium accretion where high resolution photoacoustic images of the knee joint in mice were obtained as early as one hour post-injection. Whole-body distribution was determined subsequently by labeling with ^{99m}Tc and performing tissue counting following necropsy. These studies, along with tumour imaging and in vitro binding studies, revealed that the core photoacoustic agent is also an albumin binding ligand and has a useful biological half-life and distribution profile to go along with the straightforward way it can be linked to targeting molecules.

Accordingly, the present application includes a method for detecting a presence of a target in a subject, comprising:
administering a photoacoustic agent to the subject, the photoacoustic agent comprising a photoacoustically active moiety coupled to a first bioorthogonal reactive group and a targeting entity for the target;
allowing the targeting entity to localize to the target; and
imaging the subject using photoacoustic imaging to detect the photoacoustic agent, wherein the detection of the photoacoustic agent indicates the presence of the target in the subject.

It will be appreciated by the person skilled in the art that embodiments relating to the photoacoustically active moiety and targeting entity can be varied as described herein above in relation to the photoacoustic agents. For example, in an embodiment, the photoacoustically active moiety is selected from a gold nanoparticle, a carbon nanomaterial, a polymer nanoparticle, a genetically encoded chromophore and a near infrared (NIR) dye. In another embodiment, the photoacoustically active moiety is a near infrared (NIR) dye. In another embodiment of the present application, the NIR dye is capable of binding a blood protein. In an embodiment, the blood protein is albumin.

In an embodiment, the NIR dye is a sulfonated cyanine dye.

In an embodiment, the NIR dye is of Formula (I):

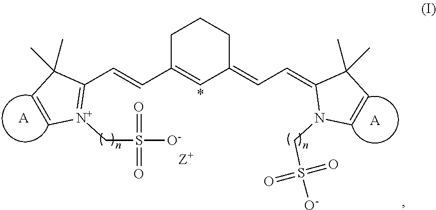

wherein

each is

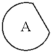

independently aryl;

each n is independently an integer of from 2 to 8;

Z⁺ is a pharmaceutically acceptable countercation; and

* is the site at which the NIR dye is coupled to the first bioorthogonal reactive group.

In an embodiment, each is

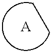

independently C₆₋₁₀aryl. In another embodiment each is

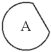

independently phenyl or naphthyl. In a further embodiment, both

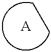

are phenyl. In another embodiment, both

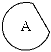

are naphthyl.

In an embodiment, each n is independently an integer of from 2 to 6. In another embodiment, each n is independently an integer of from 3 to 5. In a further embodiment of the present application both n are 4.

In an embodiment, the NIR dye is of the formula:

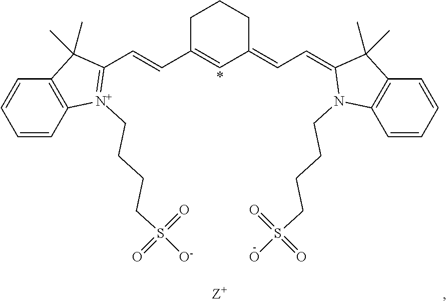

wherein

Z⁺ is a pharmaceutically acceptable countercation; and

* is the site at which the NIR dye is coupled to the first bioorthogonal reactive group.

In an embodiment, Z⁺ is Na⁺.

In an embodiment, both

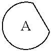

are phenyl; both n are 4; and Z⁺ is Na⁺. In another embodiment, both

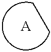

are naphthyl; both n are 4; and Z⁺ is Na⁺.

In an embodiment, the photoacoustically active moiety is coupled to the first bioorthogonal reactive group by a linker group. The linker group can be any suitable linker group. In an embodiment, the linker group is obtained by a method comprising Suzuki-Miyaura coupling between a photoacoustically active moiety having a halide (e.g. Cl) at the site designated * and a suitable diboronic acid having an aldehyde moiety in the presence of Pd(PPh₃)₄ and Cs₂CO₃, then reacting the aldehyde thereby obtained with a first bioorthogonal reactive group having a hydrazyl group under suitable conditions (e.g. conditions described in Vito et al., PLoS One 2016, 11 e0167425) to obtain the corresponding hydrazone. For example, in an embodiment, the linker group is of the formula:

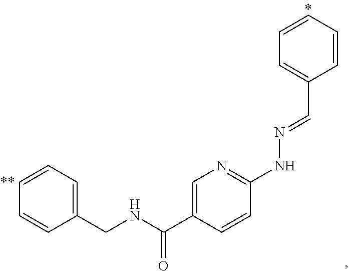 [SMILES: O=C(NCc1ccccc1)c1ccc(N/N=C/c2ccccc2)nc1]

wherein

* is the site that is bonded to the photoacoustically active dye; and

** is the site that is bonded to the first bioorthogonal reactive group.

In an another embodiment of the present application, the linker group is a suitable polyethylene glycol (PEG).

In an embodiment, the targeting entity is an antibody, a receptor ligand or a bisphosphonate. In another embodiment, the target is a cellular marker for one of inflammation, cancer, a skeletal condition, a heart abnormality, atherosclerosis, angiogenesis and intravascular thrombus formation. In an embodiment, the skeletal condition is microfractures.

In an embodiment, the targeting entity is coupled to a second bioorthogonal reactive group that is complementary to the first bioorthogonal reactive group and the first bioorthogonal reactive group and the second bioorthogonal reactive group form a bioorthogonal complex. In another embodiment, the first bioorthogonal reactive group and the second bioorthogonal reactive group, in either order, are inverse electron demand Diels Alder reaction pairs, or an azide and a functionalized phosphine, or an azide and a strained alkyne. In a further embodiment, the first bioorthogonal reactive group and the second bioorthogonal reactive group, in either order, are tetrazine and trans cyclooctene. In another embodiment, the first bioorthogonal reactive group is tetrazine and the second bioorthogonal reactive group is trans cyclooctene.

In an embodiment, the photoacoustic agent is of the formula:

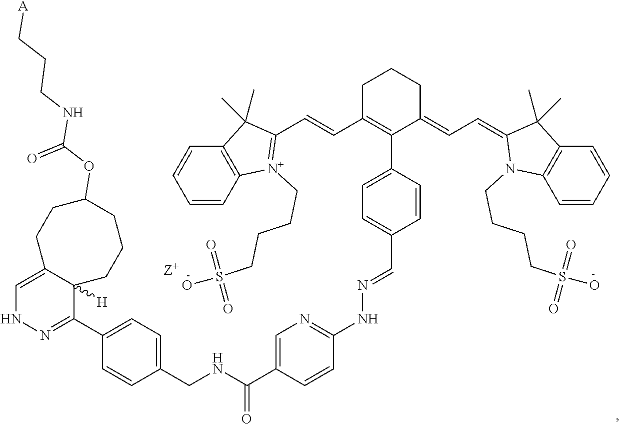

wherein

Z⁺ is a pharmaceutically acceptable countercation as defined herein; and

A is the targeting entity.

The photoacoustic agent is administered to the subject in an administrable form, for example, admixed with a pharmaceutically acceptable carrier. Examples of suitable carriers include aqueous solutions in sterile and pyrogen-free form, optionally buffered or made isotonic. In an embodiment, the carrier is distilled water, a carbohydrate-containing solution (e.g. dextrose) or a saline solution comprising sodium chloride and optionally buffered. An amount of the photoacoustic agent is administered or for use that would yield sufficient quantity of the photoacoustically active moiety for imaging purposes. In an embodiment, an amount in the range of 0.1-100 mg/kg is administered or used. In an embodiment, the administration comprises intravascular injection. Following a suitable period of time for the targeting entity to localize to the intended target site, such as a period of at least 1 hour, for example from about 12-24 hours, the subject is imaged, e.g. using ultrasound, in a region of interest to detect the presence of the photoacoustic agent. Detection of the photoacoustic agent indicates that the target is present in the subject and in some embodiments of the present application that a target disease or condition is present.

The present application also includes a method for detecting a presence of a target in a subject, comprising:
administering a targeting agent to the subject, the targeting agent comprising a targeting entity for the target coupled to a second bioorthogonal reactive group;
allowing the targeting entity to localize to the target;
administering a photoacoustic agent to the subject, the photoacoustic agent comprising a photoacoustically active moiety coupled to a first bioorthogonal reactive group that is complementary to the second bioorthogonal reactive group such that the first bioorthogonal reactive group and the second bioorthogonal reactive group form a bioorthogonal complex; and
imaging the subject using photoacoustic imaging to detect the photoacoustic agent, wherein the detection of the photoacoustic agent indicates the presence of the target in the subject.

It will be appreciated by the person skilled in the art that embodiments relating to the photoacoustically active moiety and targeting entity can be varied as suitably described herein above in relation to the photoacoustic agents. For example, in an embodiment, the photoacoustically active moiety is selected from a gold nanoparticle, a carbon nanomaterial, a polymer nanoparticle, a genetically encoded chromophore and a near infrared (NIR) dye. In another embodiment, the photoacoustically active moiety is a near infrared (NIR) dye. In another embodiment of the present application, the NIR dye is capable of binding a blood protein. In an embodiment, the blood protein is albumin.

In an embodiment, the NIR dye is a sulfonated cyanine dye.

In an embodiment, the NIR dye is of Formula (I):

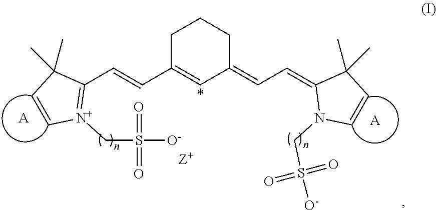

wherein

each is

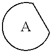

independently aryl;

each n is independently an integer of from 2 to 8;

Z⁺ is a pharmaceutically acceptable countercation; and

* is the site at which the NIR dye is coupled to the first bioorthogonal reactive group.

In an embodiment, each is

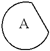

independently C₆₋₁₀aryl. In another embodiment each is

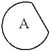

independently phenyl or naphthyl. In a further embodiment, both

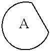

are phenyl. In another embodiment, both

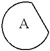

are naphthyl.

In an embodiment, each n is independently an integer of from 2 to 6. In another embodiment, each n is independently an integer of from 3 to 5. In a further embodiment of the present application both n are 4.

In an embodiment, the NIR dye is of the formula:

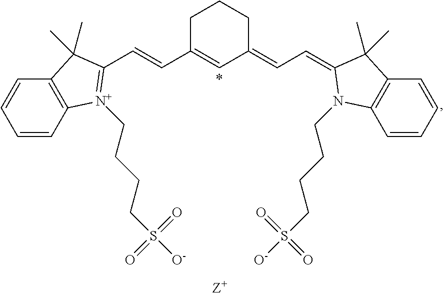

wherein

Z⁺ is a pharmaceutically acceptable countercation; and

* is the site at which the NIR dye is coupled to the first bioorthogonal reactive group.

In an embodiment, Z⁺ is Na⁺.

In an embodiment, both

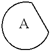

are phenyl; both n are 4; and Z⁺ is Na⁺. In another embodiment, both

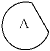

are naphthyl; both n are 4; and Z⁺ is Na⁺.

In an embodiment, the photoacoustically active moiety is coupled to the first bioorthogonal reactive group by a linker group. The linker group can be any suitable linker group. In an embodiment, the linker group is obtained by a method comprising Suzuki-Miyaura coupling between a photoacoustically active moiety having a halide (e.g. CI) at the site designated * and a suitable diboronic acid having an aldehyde moiety in the presence of Pd(PPh₃)₄ and Cs₂CO₃, then reacting the aldehyde thereby obtained with a first bioorthogonal reactive group having a hydrazyl group under suitable conditions (e.g. conditions described in Vito et al., PLoS One 2016, 11 e0167425) to obtain the corresponding hydrazone. For example, in an embodiment, the linker group is of the formula:

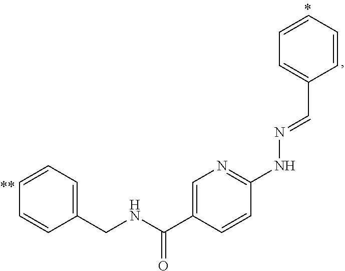 [SMILES: O=C(NCc1ccccc1)c1ccc(N/N=C/c2ccccc2)nc1]

wherein

* is the site that is bonded to the photoacoustically active dye; and

** is the site that is bonded to the first bioorthogonal reactive group.

In an another embodiment of the present application, the linker group is a suitable polyethylene glycol (PEG).

In an embodiment, the first bioorthogonal reactive group and the second bioorthogonal reactive group, in either order, are inverse electron demand Diels Alder reaction pairs, or an azide and a functionalized phosphine, or an azide and a strained alkyne. In another embodiment, the first bioorthogonal reactive group and the second bioorthogonal reactive group, in either order, are tetrazine and trans cyclooctene. In a further embodiment, the first bioorthogonal reactive group is tetrazine and the second bioorthogonal reactive group is trans cyclooctene.

In an embodiment, the targeting entity is an antibody, a receptor ligand or a bisphosphonate. In another embodiment, the target is a cellular marker for one of inflammation, cancer, a skeletal condition, a heart abnormality, atherosclerosis, angiogenesis and intravascular thrombus formation. In an embodiment, the skeletal condition is microfractures.

In an embodiment, the bioorthogonal complex is of the formula:

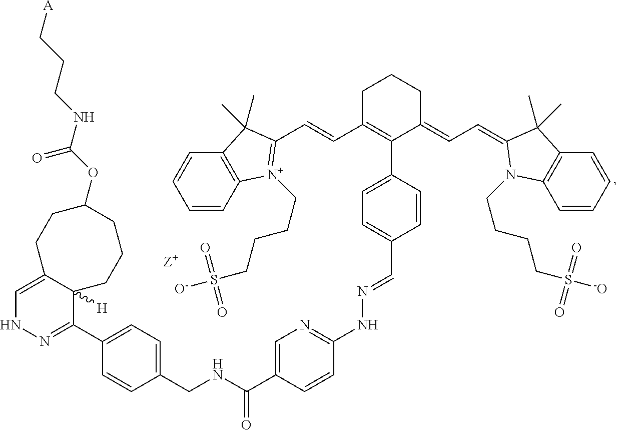

wherein

Z⁺ is a pharmaceutically acceptable countercation as defined herein; and

A is the targeting entity.

The photoacoustic agent and the targeting agent are administered to the subject in an administrable form, for example, admixed with a pharmaceutically acceptable carrier. Examples of suitable carriers include aqueous solutions in sterile and pyrogen-free form, optionally buffered or made isotonic. In an embodiment, the carrier is distilled water, a carbohydrate-containing solution (e.g. dextrose) or a saline solution comprising sodium chloride and optionally buffered. An amount of the photoacoustic agent and targeting agent is administered or for use that would yield sufficient quantity of a bioorthogonal complex for imaging purposes. In an embodiment, an amount in the range of 0.1-100 mg/kg of the targeting agent is administered or used. In an embodiment, the administration comprises intravascular injection. Following a suitable period of time for the targeting entity to localize to the intended target site, such as a period of at least 1 hour, for example from about 12-24 hours, the photoacoustic agent is administered to the subject. Following administration of the photoacoustic agent in an amount suitable to react with the targeting agent, and a suitable amount of time for the photoacoustic agent to localize and for the bioorthogonal groups to react, e.g. a period of about 2-30 minutes, or about 4-10 minutes, the subject is imaged, e.g. using ultrasound, in a region of interest to detect the presence of the photoacoustic agent. Detection of the photoacoustic agent indicates that the target is present in the subject and in some embodiments of the present application that a target disease or condition is present.

The present application also includes a composition comprising one or more photoacoustic agents of the present application and a carrier. In an embodiment, the composition is a pharmaceutical composition comprising one or more photoacoustic agents of the present application and a pharmaceutically acceptable carrier. In an embodiment of the present application, the pharmaceutical composition is formulated for intravenous injection.

The present application also includes a composition comprising one or more targeting agents of the present application and a carrier. In an embodiment, the composition is a pharmaceutical composition comprising one or more targeting agents of the present application and a pharmaceutically acceptable carrier. In an embodiment of the present application, the pharmaceutical composition is formulated for intravenous injection.

The present application also includes a kit comprising:
a photoacoustic agent, the photoacoustic agent comprising a photoacoustically active moiety coupled to a first bioorthogonal reactive group;
a targeting agent, the targeting agent comprising a targeting entity for a target coupled to a second bioorthogonal reactive group that is complementary to the first bioorthogonal reactive group such that the first bioorthogonal reactive group and the second bioorthogonal reactive group form a bioorthogonal complex; and
optionally instructions for use of the targeting agent and the photoacoustic agent in imaging a subject using photoacoustic imaging to detect the photoacoustic agent.

It will be appreciated by the person skilled in the art that embodiments relating to the photoacoustically active moiety and targeting entity can be varied as suitably described herein above in relation to the photoacoustic agents. For example, in an embodiment, the photoacoustically active moiety is selected from a gold nanoparticle, a carbon nanomaterial, a polymer nanoparticle, a genetically encoded chromophore and a near infrared (NIR) dye. In another embodiment, the photoacoustically active moiety is a near infrared (NIR) dye. In another embodiment of the present application, the NIR dye is capable of binding a blood protein. In an embodiment, the blood protein is albumin.

In an embodiment, the NIR dye is a sulfonated cyanine dye.

In an embodiment, the NIR dye is of Formula (I):

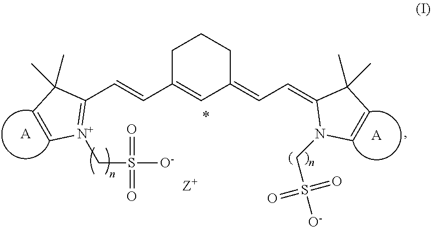

wherein

each is

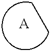

independently aryl;

each n is independently an integer of from 2 to 8;

Z⁺ is a pharmaceutically acceptable countercation; and

* is the site at which the NIR dye is coupled to the first bioorthogonal reactive group.

In an embodiment, each is

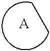

independently C₆₋₁₀aryl. In another embodiment each is

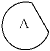

independently phenyl or naphthyl. In a further embodiment, both

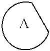

are phenyl. In another embodiment, both

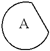

are naphthyl.

In an embodiment, each n is independently an integer of from 2 to 6. In another embodiment, each n is independently an integer of from 3 to 5. In a further embodiment of the present application both n are 4.

In an embodiment, the NIR dye is of the formula:

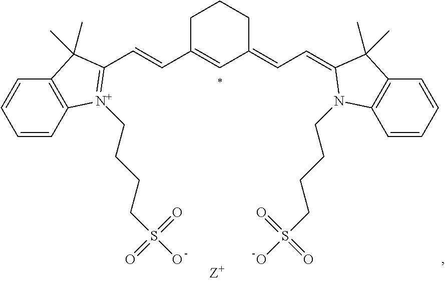

wherein

Z⁺ is a pharmaceutically acceptable countercation; and

* is the site at which the NIR dye is coupled to the first bioorthogonal reactive group.

In an embodiment, Z⁺ is Na⁺.

In an embodiment, both

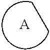

are phenyl; both n are 4; and Z⁺ is Na⁺. In another embodiment, both

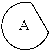

are naphthyl; both n are 4; and Z⁺ is Na⁺.

In an embodiment, the photoacoustically active moiety is coupled to the first bioorthogonal reactive group by a linker group. The linker group can be any suitable linker group. In an embodiment, the linker group is obtained by a method comprising Suzuki-Miyaura coupling between a photoacoustically active moiety having a halide (e.g. CI) at the site designated * and a suitable diboronic acid having an aldehyde moiety in the presence of Pd(PPh₃)₄ and Cs₂CO₃, then reacting the aldehyde thereby obtained with a first bioorthogonal reactive group having a hydrazyl group under suitable conditions (e.g. conditions described in Vito et al., PLoS One 2016, 11 e0167425) to obtain the corresponding hydrazone. For example, in an embodiment, the linker group is of the formula:

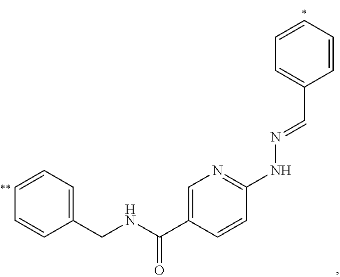 [SMILES: O=C(NCc1ccccc1)c1ccc(N/N=C/c2ccccc2)nc1]

wherein

* is the site that is bonded to the photoacoustically active dye; and

** is the site that is bonded to the first bioorthogonal reactive group.

In an another embodiment of the present application, the linker group is a suitable polyethylene glycol (PEG).

In an embodiment, the first bioorthogonal reactive group and the second bioorthogonal reactive group, in either order, are inverse electron demand Diels Alder reaction pairs, or an azide and a functionalized phosphine, or an azide and a strained alkyne. In another embodiment, the first bioorthogonal reactive group and the second bioorthogonal reactive group, in either order, are tetrazine and trans cyclooctene. In a further embodiment, the first bioorthogonal reactive group is tetrazine and the second bioorthogonal reactive group is trans cyclooctene.

In an embodiment, the targeting entity is an antibody, a receptor ligand or a bisphosphonate. In another embodiment, the target is a cellular marker for one of inflammation, cancer, a skeletal condition, a heart abnormality, atherosclerosis, angiogenesis and intravascular thrombus formation. In an embodiment, the skeletal condition is microfractures.

In an embodiment, the targeting agent and the photoacoustic agent are capable of reacting to form a bioorthogonal complex of the formula:

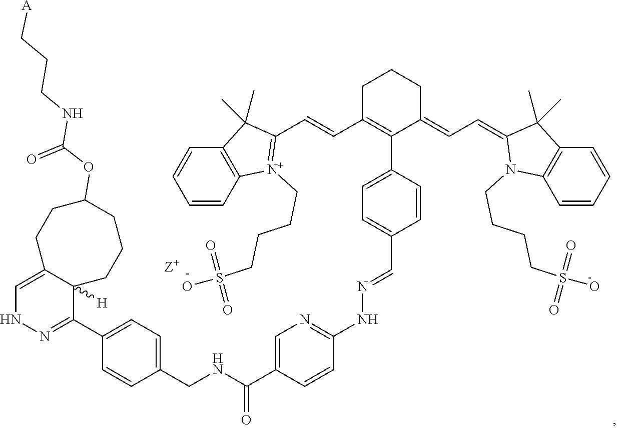

wherein

Z⁺ is a pharmaceutically acceptable countercation as defined herein; and

A is the targeting entity.

In an embodiment, the kits further comprise other reagents for performing photoacoustic imaging, such as buffers, syringes and the like.

The following non-limiting examples are illustrative of the present application:

### EXAMPLES

### Example 1: An Albumin Binding Tetrazine-Derived Near-Infrared Dye for Targeted Photoacoustic Imaging

I. Materials and Methods

Unless otherwise noted, all reagents and solvents were ACS grade and were purchased from commercial suppliers and used without further purification. 6-(2-(tert-butoxycarbonyl)hydrazinyl)nicotinic acid was purchased from Ontario Chemicals Inc. (E)-cyclooct-4-enol (TCO-OH), (E)-4-cycloocten-1-yl-2,5-dioxo-1-pyrrolidinyl ester carbonic acid (TCO-NHS), and (4-(1,2,4,5-tetrazin-3-yl)phenyl)methanamine hydrochloride were purchased from Conju-Probe. N,N-dimethylformamide, and methanol were purchased from Caledon. Distilled water was used for all experiments. Deuterated solvents for NMR samples were purchased from Cambridge Isotope Laboratories. Sodium borate and potassium sodium tartrate (KNaC₄H₄O₆.4H₂O) were purchased from Anachemia Canada-VWR. Sodium carbonate was purchased from EM Science. All other compounds were purchased from Sigma-Aldrich. ^{99m}Tc was obtained as TcO₄⁻ from a ⁹⁹Mo/^{99m}Tc generator (Lantheus Medical Imaging) in saline (0.9% NaCl). Caution: ^{99m}Tc is a γ-emitter (E_{γ}=140 keV, t_{1/2}=6 h) and should only be used in a licensed and appropriately shielded facility.

¹H and ¹³C NMR spectra were recorded on a Bruker Avance AV-600 instrument at 300 K. High Resolution Mass Spectra (HRMS) were collected on a Waters/Micromass Q-Tof Global Ultima spectrometer. Microwave reactions were performed using a Biotage Initiator 60 instrument. High Performance Liquid Chromatography (HPLC) was performed using a Phenomenex Polar-RP column (4 μm, 250×10 mm) operating at a flow rate of 4.0 mL/min was used with a Waters 1525 system connected to a Bioscan γ detector and a 2998 photodiode array detector monitoring at 350 nm and 700 nm operated using the Empower software package. Flow rates were 1 mL/min. (analytical) or 4 mL/min. (preparative) using the following methods: Method 1 (Solvent A: H₂O+0.1% trifluoroacetic acid (TFA), Solvent B: Acetonitrile+0.1% TFA) 0-1 min 90% B, 1-20 min 10% B, 20-21 min 10-90% B). Solvents were evaporated using either a Biotage V10 system or under reduced pressure with a rotary evaporator. Compounds were dried using a VirTis Benchtop lyophilizer equipped with an Edwards RV5 pump.

Cells and Culture Methods.

143B (CRL-8303) cells were purchased from ATCC. 143B (CRL-8303) cells were cultured in minimum essential media, non-essential amino acid solution (MEM, NEAA) no glutamine, 0.015 mg/mL 5-bromo-2′-deoxyuridine supplemented with 10% fetal bovine serum (FBS), and 1% penicillin streptomycin. The cell line was maintained at 37° C. under 5% CO₂.

Animal Care.

Female BALB/c immunocompetent mice (Charles River Laboratories, Kingston, N.Y.) at 4-6 weeks of age were sterile housed and maintained at 24° C. with a 12 h light/dark cycle and were provided autoclaved food and water ad libitum. All procedures were conducted according to the guidelines of the Committee for Research and Ethical Issues of the International Association for the Study of Pain, and guidelines established by the Canadian Council on Animal Care and the McMaster University Animal Research Ethics Board. Female 10-11 week old BALB/c nu/nu mice (Charles River Laboratories, Kingston, N.Y.) were injected with 2.0×10⁶ 143 B cells in Matrigel:Dulbecco's phosphate-buffered saline (DPBS) (1:1) subcutaneously into the right flank. Photoacoustic studies were performed 10 days following tumor inoculation.

Biodistribution Studies.

Biodistribution studies were performed using female BALB/c mice (Charles River, Kingston, N.Y.) at the indicated time points. The mice were administered the agents via tail vein injection with the final volume not exceeding 200 μL. A 5 mg/mL solution of compound 9 (see Scheme 2, below) in 10% ethanol in saline was administered at a dose of 20 mg/kg to all mice (approximately 518 kBq of 9). For all studies, at 1, 3, 6, or 24 h post-injection of the labeled compound, the animals were euthanized by cervical dislocation. Fluids, bone (knee and shoulder), and select tissues were collected, weighed, and counted in a PerkinElmer Wizard 1470 automatic gamma counter. Decay correction was used to normalize organ activity measurements to time of dose preparation for data calculations. Data is expressed as percent injected dose per gram tissue or fluid (% ID/g) or percent injected dose per organ (% ID/O).

Photoacoustic Imaging Studies.

Photoacoustic imaging was performed using Vevo LAZR-X (FUJIFILM VisualSonics Inc., Toronto, ON, Canada) Imaging system equipped with a 680-970 nm laser. Mice were anaesthetized with isofluorane and set up on a platform that monitored the respiration rate and the heart rate of the mouse. The hair on the hindlimb was removed while the mouse was anaesthetized. A 30-MHz, linear array ultrasound transducer with integrated fiber optic light delivery (LZ-400 and MX-400, FUJIFILM VisualSonics, Inc.) was positioned laterally overtop the hindlimb knee joint. The integrated fiber bundle delivered 15 to 20 mJ/cm² of light to the hindlimb of the mouse.

II. Synthetic Procedures

Synthesis of sodium 4-((Z)-2-((E)-2-(6-((E)-2-(3,3-dimethyl-1-(4-sulfonatobutyl)-3H-indol-1-ium-2-yl)vinyl)-4′-formyl-4,5-dihydro-[1,1′-biphenyl]-2(3H)-ylidene)ethylidene)-3,3-dimethylindolin-1-yl)butane-1-sulfonate (Compound 3) In a round bottom flask, IR-783 (80 mg, 0.11 mmol), 3-formylboronic acid (31 mg, 0.21 mmol), Pd(PPh₃)₄ (10 mg, 8 mol %), and Cs₂CO₃ (20 mg, 50 mol %) were dissolved in water (10 mL) and heated at 100° C. for 4 hours. The product was then dried and purified by HPLC (Method 1) yielding a teal solid. Yield: (71 mg, 96%)¹H NMR (600 MHz, (CD₃)₂SO): δ (ppm) 10.22 (s, 1H), 8.18 (d, 2H), 7.56 (d, 2H), 7.45 (d, 2H), 7.35 (m, 4H), 7.16 (d, 2H), 6.99 (d, 2H), 6.28 (d, 2H), 4.13 (t, 4H), 2.72 (t, 5H), 1.98 (t, 2H), 1.74 (dd, 9H), 1.08 (d, 14H). ¹³C NMR (150 MHz, (CD₃)₂SO): δ (ppm) 193.48, 171.68, 159.78, 147.08, 146.06, 142.55, 141.11, 136.14, 130.99, 130.79, 130.09, 128.93, 125.10, 122.85, 111.65, 100.91, 51.24, 48.65, 43.92, 27.46, 26.54, 24.57, 22.96, 21.30. HRMS (ES⁻) m/z calculated for C₄₅H₅₁N₂O₇S₂: 795.3143 [M-H⁻], found 795.3143.

Synthesis of hydrozone (Compound 5) from Compound 3 and hydrazinonicotinamide tetrazine In a round bottomed flask 3 (90 mg, 0.113 mmol), hydrazinonicotinamide (HYNIC)-tetrazine (44 mg, 0.136 mmol), and aniline (62 μL, 0.681 mmol) were added with 10 mL absolute ethanol and the flask was sealed. This mixture was allowed to stir at room temperature (RT) for 12 h. The product was isolated by HPLC Method 1, dried under vacuum yielding a green solid. Yield: 30 mg (35%). ¹H NMR ((CD₃OD), 600 MHz): δ (ppm) 10.33 (s, 1H), 8.67 (d, 1H), 8.59 (d, 2H), 8.54 (dd, 1H), 8.45 (s, 1H), 8.23 (d, 2H), 7.66 (d, 2H), 7.43 (d, 2H), 7.34 (m, 2H), 7.29 (d, 2H), 7.26 (d, 2H), 7.23 (s, 1H), 7.16 (t, 2H), 6.23 (d, 2H), 4.09 (t, 4H), 3.20 (m, 2H), 2.84 (t, 4H), 2.76 (t, 4H), 2.07 (m, 2H), 1.93 (s, 2H), 1.87 (m, 7H), 1.31 (t, 4H), 1.22 (s, 12H). ¹³C NMR ((CD₃)₂SO), 150 MHz): δ (ppm) 171.09, 165.42, 158.08, 142.11, 140.61, 132.02, 131.49, 131.43, 130.74, 130.32, 129.78, 128.78, 128.70, 128.40, 128.18, 128.08, 127.81, 100.22, 50.76, 28.09, 27.06, 26.03, 22.51. HRMS (ES⁻) m/z calculated for C₆₀H₆₃N₁₀O₇S₂: 1099.4323 [M-H⁻], found 1099.4289.

III. Results and Discussion

The preparation of functionalized cyanine dyes is non-trivial and often low yielding. Consequently, for general utility and accessibility, an efficient synthetic route to the target tetrazine is desirable (Scheme 1).

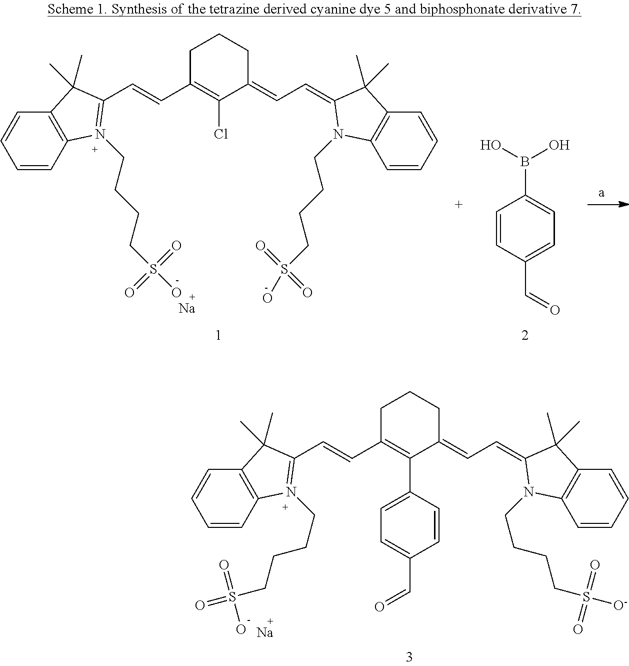

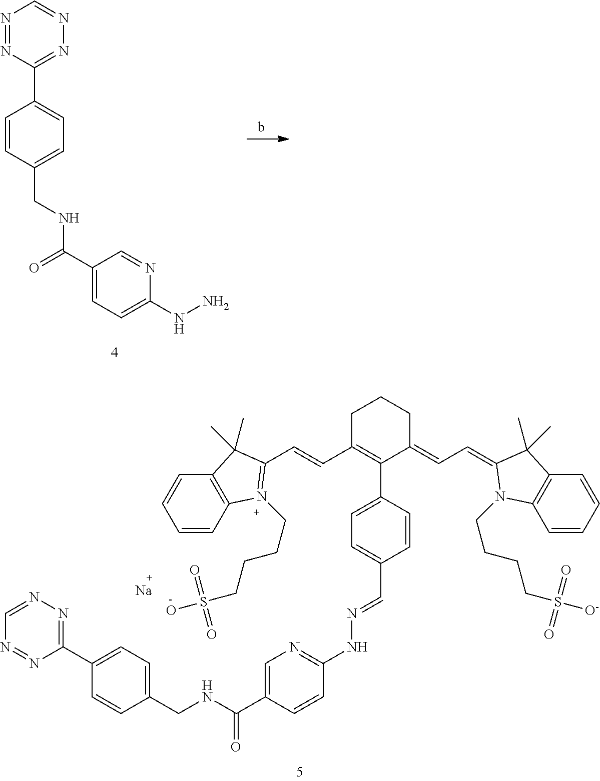

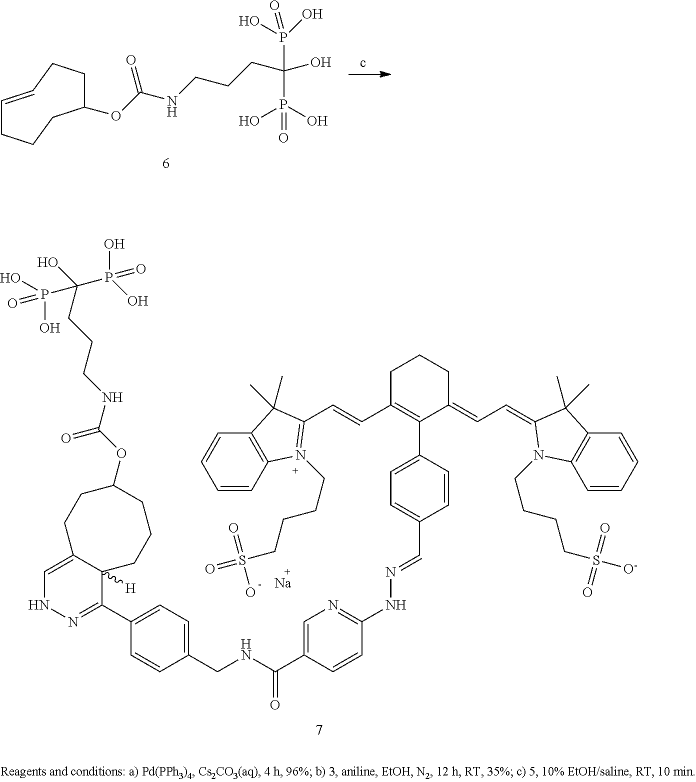

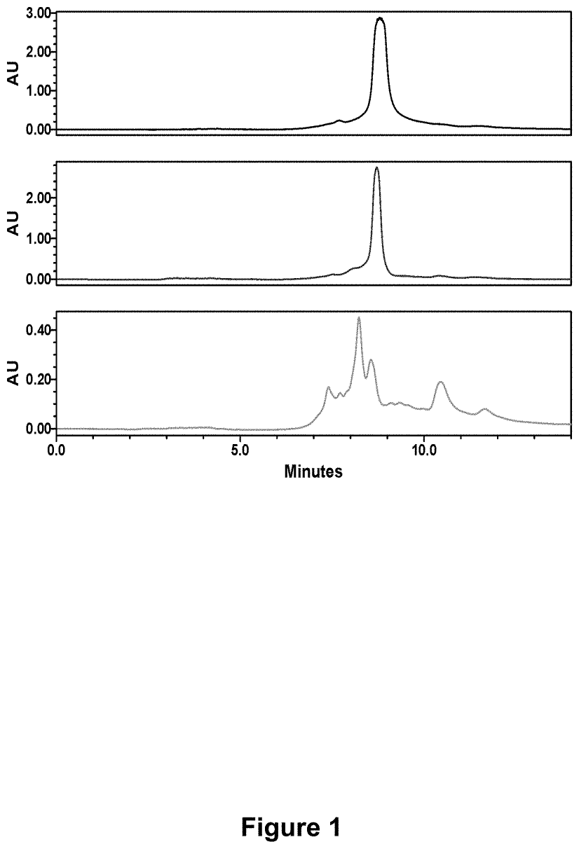

Compound 5 was prepared from the commercially available dye IR-783, which is known to produce a strong photoacoustic signal, in only two steps. The first step involved the preparation of the IR-783-aldehyde 3 which was done successfully using a Suzuki-Miyaura coupling between 1 and 2 in the presence of Pd(PPh₃)₄ and Cs₂CO₃. A significant number of different catalysts, bases and reaction conditions were screened to maximize the yield of 3 (96%). The aldehyde was subsequently coupled to tetrazine 4, prepared using a literature procedure,¹⁴ to form the hydrazone 5. Following characterization by nuclear magnetic resonance (NMR) spectroscopy, high resolution mass spectrometry (HRMS) and high performance liquid chromatography (HPLC), the stability of 5 in plasma was evaluated. The dye-tetrazine conjugate showed no signs of degradation out to 24 h in murine plasma at 37° C. (FIG. 1) and the tetrazine remained reactive towards trans-cyclooctene (TCO)-derivatives during that time.

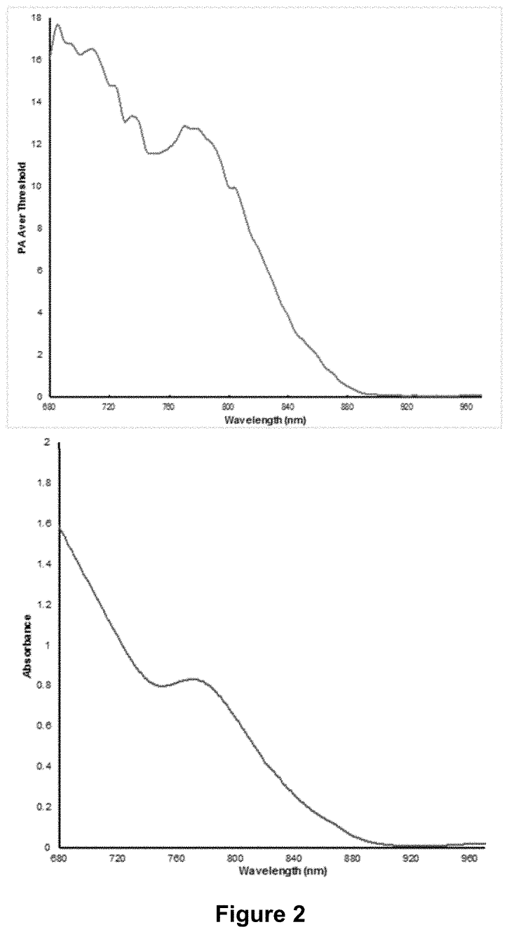

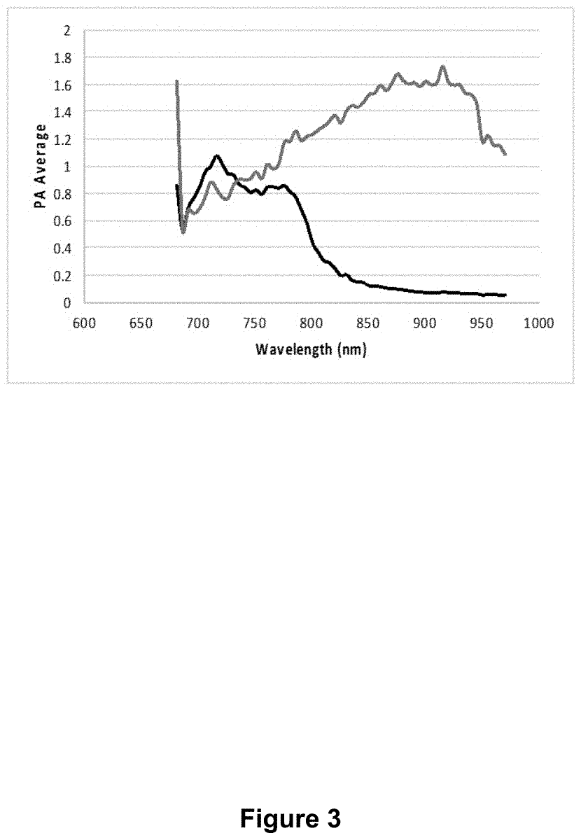

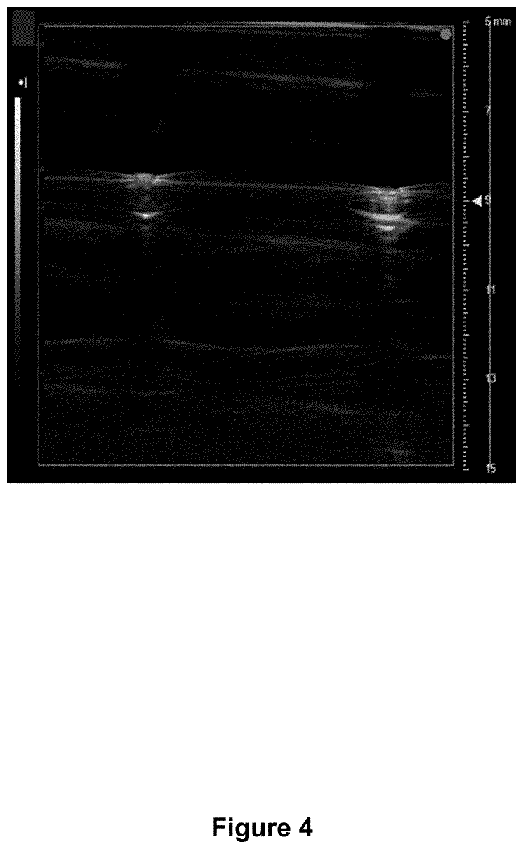

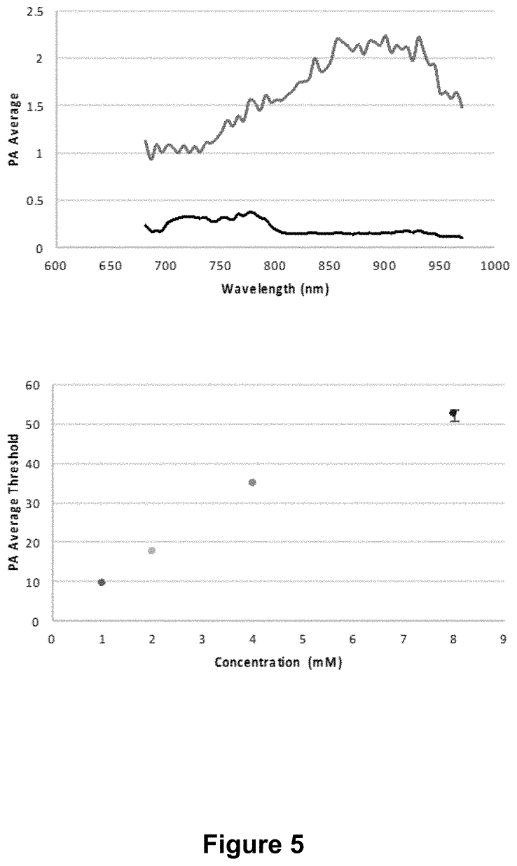

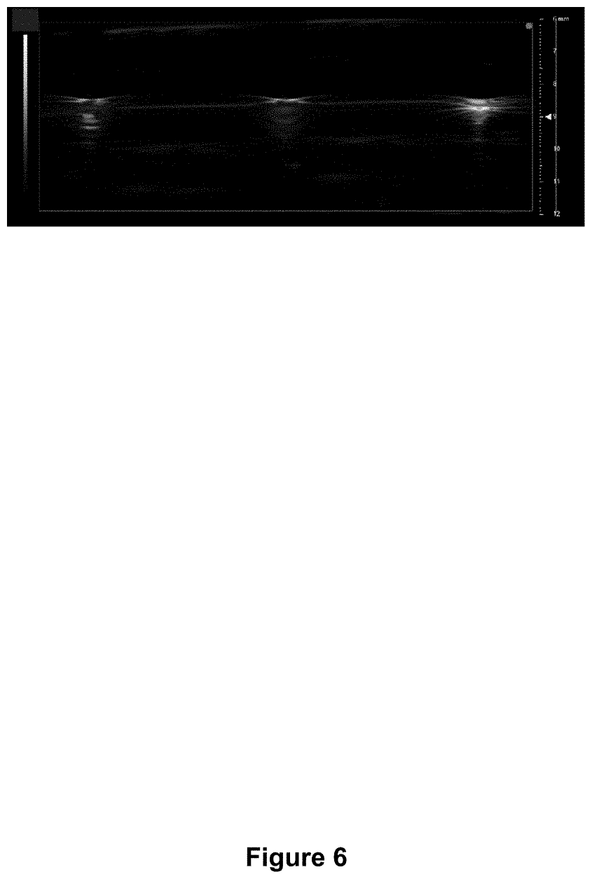

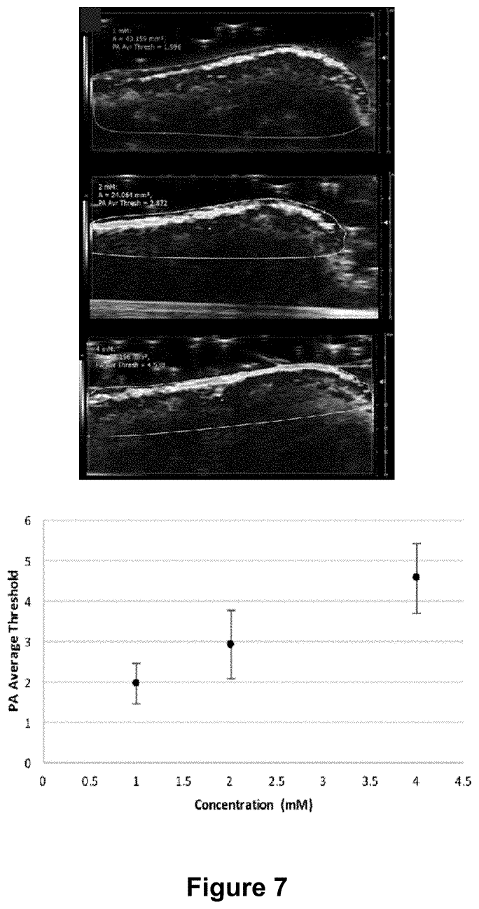

Photoacoustic properties of 5 in saline were assessed initially in a Vevo Phantom imaging chamber (Fujifilm VisualSonics Inc., Toronto) scanning over a wavelength range of 680-970 nm. The photoacoustic spectrum (FIG. 2; upper spectrum) and absorbance spectrum (FIG. 2; lower spectrum) of 5 were nearly identical where the optimal wavelength for imaging was 775 nm, which should be distinguishable from the photoacoustic signals arising from oxygenated and deoxygenated hemoglobin (684, 750 and 870 nm). To test this, the photoacoustic spectra of 5 and a TCO-conjugate 7 (vide infra) in saline (0.1 mM), and blood (0.1 mM) were compared to that for whole blood alone (FIG. 3). It was evident that the dyes were readily detected in both saline and blood (shown in green in color images) and not in the control sample of blood alone (FIG. 4). Interestingly, the intensity of the image in blood alone was greater than in saline, which while not wishing to be limited by theory, is likely due to binding to albumin; a phenomenon which was investigated in more detail following in vivo studies. The photoacoustic signal was measured in triplicate over a range of concentrations which showed increasing intensity with increasing amounts of 5 (FIGS. 5-6) where photoacoustic average threshold is the average of the top 10% of the signal. This was evident visually when cotton swabs were dipped in increasing concentrations of 7 and then imaged on the photoacoustic imaging scanner (FIG. 7).¹⁵

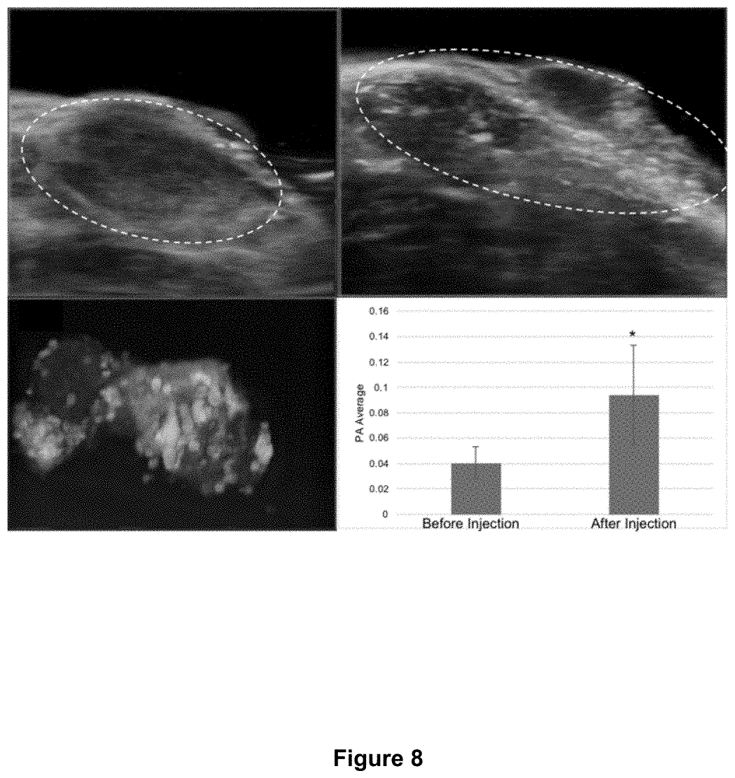

Prior to developing and testing a targeted derivative, in vivo imaging studies were performed with 5 and 143B human osteosarcoma xenografts. Compound 5 was soluble in 10% ethanol in saline and formulated at a concentration of 5 mg mL⁻¹, which conveniently is higher than what can be achieved for ICG (1 mg mL⁻¹). Prior to injection, tumors were imaged in order to separate the signals arising from oxygenated and deoxygenated hemoglobin, and 5, which are shown in red, blue and green respectively in color images of FIG. 8. Following administration of 200 μL of 5 (1 mg), signal arising from 5 was clearly visible in the tumor as early as 1 hour post-injection (FIG. 8, upper right image). Mice were sacrificed and the tumors were imaged ex vivo (FIG. 8, lower left image) where uptake of the dye within the tumor was clearly evident. Analysis of the signal intensity from the in vivo images showed a 2.3× increase in signal intensity in the tumor at 1 hour post injection versus the background signal in the tumor before the injection (FIG. 8, lower right plot).

There are a number of TCO-derived molecules, including antibodies and small molecules, that are available for use as targeting vectors.^{16,17,18,19} For a proof of concept study, compound 5 was combined with a TCO-derived bisphosphonate (TCO-BP (6)). TCO-BP has been used to target radiolabeled tetrazines to sites of high calcium accretion. Conveniently, the compound allows testing to be performed in inexpensive healthy mouse models since tetrazine derivatives coupled to TCO-BP should localize to elbow and knee joints where there is elevated turnover of calcium.^{16,20,21} Compound 7 was prepared by mixing compounds 5 and 6 at room temperature for 10 min in quantitative yield; highlighting the ease with which new photoacoustic imaging agents can be prepared using this approach.

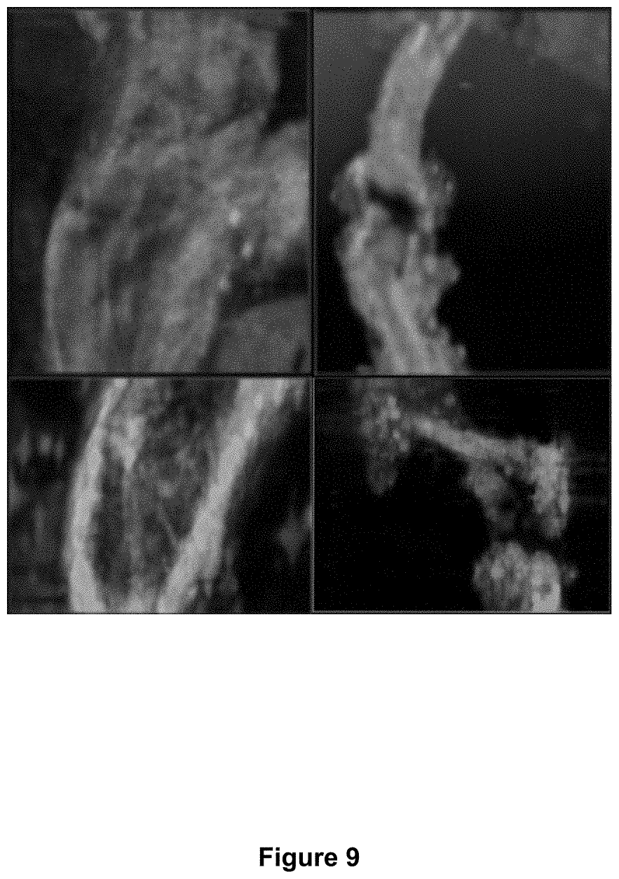

For in vivo studies, a group of Balb/c mice were administered the parent tetrazine 5 and a second group received the bisphosphonate derivative 7. Imaging of the knee was performed for both groups at 1 hour post-injection where the same timepoint was used in studies using radiolabeled tetrazines and TCO-BP.¹⁶ The photoacoustic images of animals administered 5 did not show any significant signal in the knee or shoulder joints, which was expected given that the dye is not targeted to bone. In contrast, mice given compound 7 showed a significant signal in the knee joints (FIG. 9). Images taken of the leg ex vivo confirmed the results where intense uptake was observed only for animals who were treated with 7.

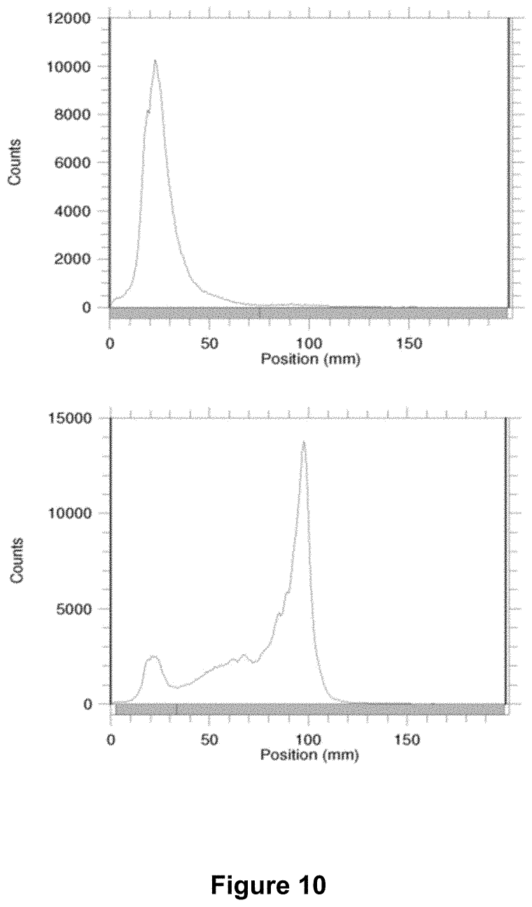

An additional benefit to using TCO-BP is that the bisphosphonate can be labeled with ^{99m}Tc (8) and the product coupled to 5 so that the whole-body biodistribution of the photoacoustic imaging dye can be determined quantitatively. ^{99m}Tc labeling of TCO-BP was performed following a literature procedure¹⁶ where radio-TLC (FIG. 10, Table 1) showed the presence of the product and a small amount of pertechnetate.

TABLE 1
Region analysis of RadioTLC traces of FIG. 10

| | | | | | | | | |
|---|---|---|---|---|---|---|---|---|
| | (mm) | (mm) | (mm) | | Region | Region | % of | % of |
| Reg | Start | Stop | Centroid | RF | Counts | CPM | Total | ROI |

Acetonitrile

| | | | | | | | | |
|---|---|---|---|---|---|---|---|---|
| Rgn 1 | −0.2 | 75.2 | 26.0 | 0.130 | 203903.0 | 67967.7 | 97.34 | 97.34 |
| Rgn 2 | 75.2 | 199.1 | 130.7 | 0.518 | 5573.0 | 1857.7 | 2.66 | 2.66 |

| | | | | |
|---|---|---|---|---|
| 2 Peaks | 209476.0 | 69825.3 | 100.00 | 100.00 |

Water

| | | | | | | | | |
|---|---|---|---|---|---|---|---|---|
| Rgn 1 | 2.3 | 33.3 | 21.8 | 0.109 | 40067.0 | 13355.7 | 10.99 | 11.00 |
| Rgn 2 | 33.3 | 199.1 | 83.8 | 0.419 | 324197.0 | 108065.7 | 88.96 | 89.00 |

| | | | | |
|---|---|---|---|---|
| 2 Peaks | 364264.0 | 121421.3 | 99.96 | 100.00 |

Compound 5 was then added to 8 and the solution allowed to mix at room temperature for 20 min giving 9 in high yield (>99%) (Scheme 2).

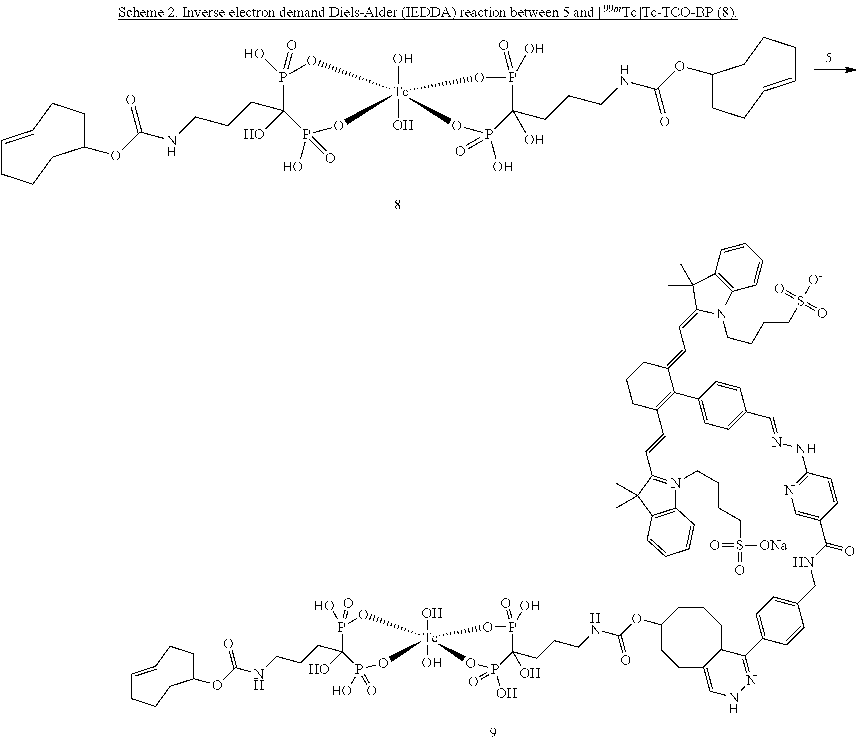

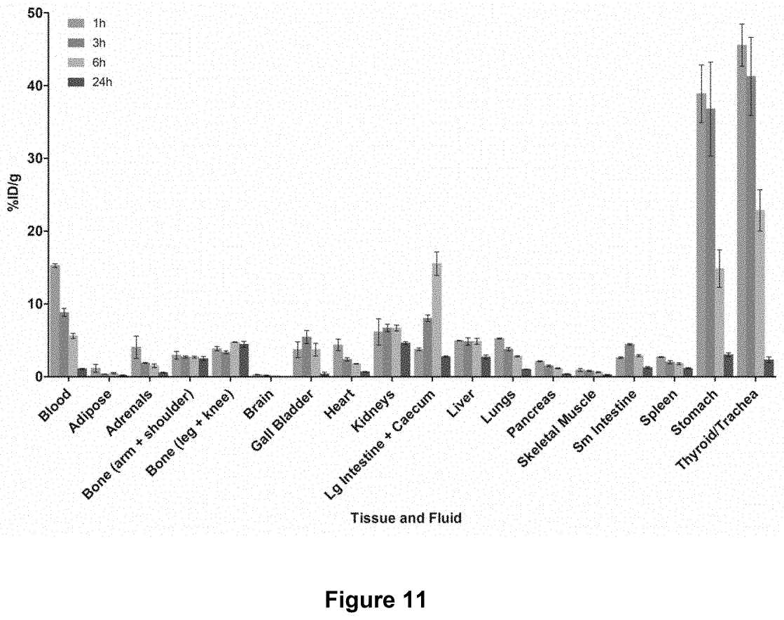

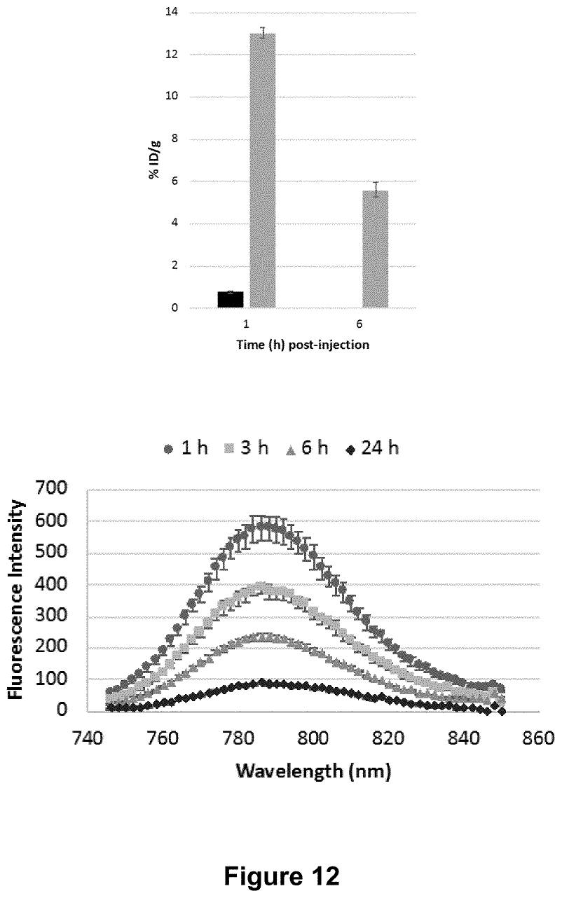

Mice were administered 9 (518 kBq) and sacrificed at 1, 6, and 24 h post injection where fluids, bone (knee, shoulder), and other key tissues were collected, weighed, and counted in a gamma counter. The resulting data is shown in FIG. 11 and expressed as percent injected dose per gram of tissue or fluid (% ID g⁻¹). The shoulder and knee showed 3-5% ID g⁻¹ (6-10% ID) which remained constant over 24 hours. The compound also exhibited some level of localization in all major tissues and organs with high uptake in the thyroid and stomach being associated with the presence of pertechnetate. Activity was detected in both the kidneys and liver suggesting, while not wishing to be limited by theory, that dual routes of excretion are likely. Surprisingly, at the 1 h timepoint, over 15% ID g⁻¹ was found in the blood, which at 1 h was 20× higher than that observed for [^{99m}Tc]Tc-TCO-BP alone (0.78% ID g⁻¹) and 187× higher at 6 h (FIG. 12, Table 2).

TABLE 2

| Time | Fl @ 786 | Amount in blood | % of total amount |
|---|---|---|---|
| (h) | nm | (μmol) | injected |
| 1 | 580 ± 39 | 0.025 | 13.7% |
| 3 | 388 ± 17 | 0.017 | 9.3% |
| 6 | 234 ± 11 | 0.011 | 6.0% |
| 24 | 88 ± 2.8 | 0.005 | 2.7% |

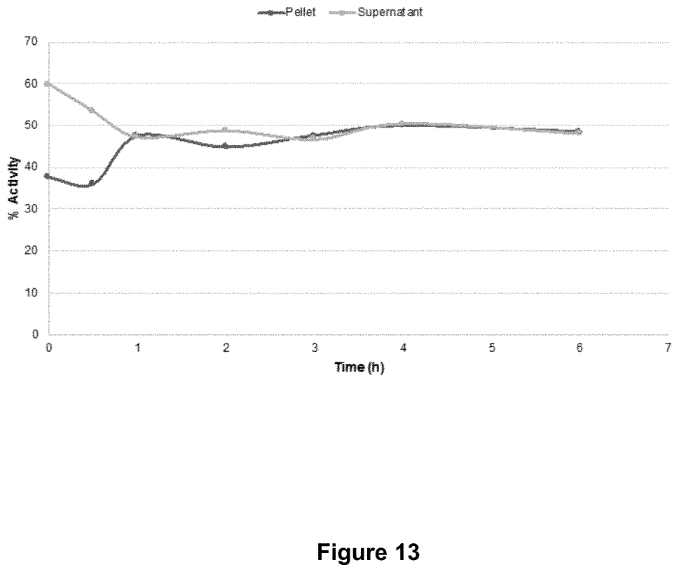

Based on the high blood concentrations, a subsequent plasma binding study was performed on 9 which showed 50% of the activity was present in the pellet containing the proteins and 50% in the supernatant after 1 hour (FIG. 13, Table 3). A parallel study was also performed where the fluorescence intensity arising from the cyanine dye in blood following administration of 9 to the Balb/c mice was measured at 1, 6, and 24 h. The blood half-life of the dye (3.8 h) was similar to that determined using radioactive counting (3.2 h) where, while not wishing to be limited by theory, the small difference is likely due to dissociation of the ^{99m}Tc, which is known to happen in vivo particularly at the modest concentrations of ligand used in this study.

TABLE 3

| Time (h) | Pellet | Supernatant |
|---|---|---|
| 0 | 32.79 | 52.00 |
| 0.5 | 36.10 | 53.44 |
| 1 | 47.53 | 47.27 |
| 2 | 45.29 | 49.35 |
| 3 | 47.72 | 46.82 |
| 4 | 46.88 | 47.42 |
| 6 | 47.83 | 47.46 |

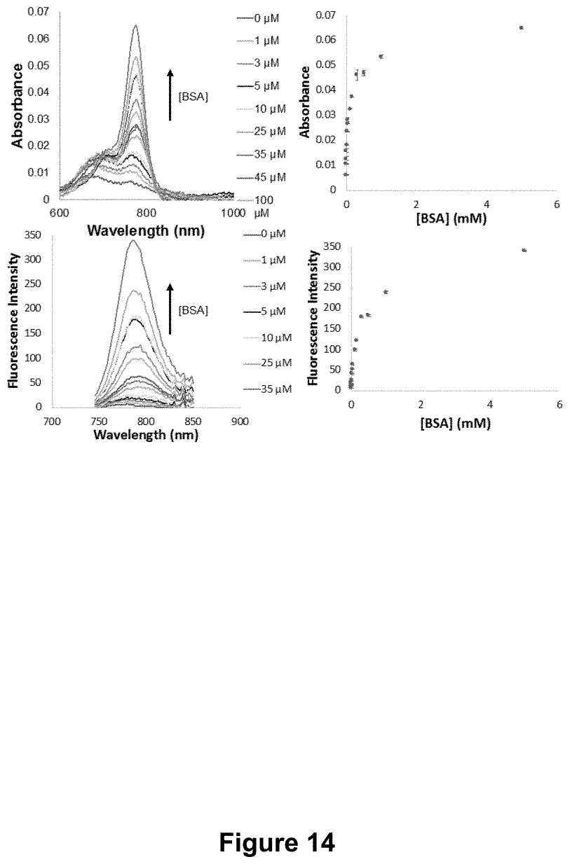

There is significant interest in bifunctional molecules that bind blood proteins such as albumin, for example, as a way to prolong circulation of drugs that are rapidly excreted.²² Sulfonated cyanine dyes have been shown to non-covalently bind to the hydrophobic pockets of albumin.²³ For the tetrazine-IR783 derivative, a preliminary albumin binding study was performed looking at the absorbance and emission of 5 with varying concentrations of BSA. The absorbance spectrum (FIG. 14) exhibited a band around 650 nm, which is indicative of H-band aggregates with stacked dipoles.²⁴ Upon the addition of albumin, the absorbance maxima shifts to that for a dimer, seen at 700 nm, and a monomer (774 nm) which is indicative of disaggregation. When experiments were performed at high dye concentrations, aggregation completely quenches the fluorescence signal, which can be restored through the addition of albumin. While not wishing to be limited by theory, this is indicative of cut-on fluorescence and the dye binding to a hydrophobic binding site of albumin.³⁴

In summary, a new tetrazine-derived cyanine dye that can be used to create targeted photoacoustic imaging probes through IEEDA chemistry was prepared. The ability to rapidly and effectively couple the dye to a TCO-derivative was demonstrated using a TCO-bisphosphonate and a ^{99m}Tc labeled analogue where the product was used to visualize the knee joint of mice. While active targeting was used in this example, there is also the opportunity to employ pre-targeting strategies, for example, where a TCO-derivative is administered first, and once useful target uptake achieved, the tetrazine can be injected, localizing at the site of interest through IEEDA coupling in vivo. This approach would benefit from the pharmacokinetics and stability observed for 5.

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE DESCRIPTION

¹ L. V Wang, J. Yao, Nat. Methods 2016, 13, DOI 10.1038/nmeth.3925.
² A. Needles, A. Heinmiller, J. Sun, C. Theodoropoulos, D. Bates, D. Hirson, M. Yin, F. S. Foster, IEEE Trans. Ultrason. Ferroelectr. Freq. Control 2013, 60, 888-897.
³ A. Bar-zion, M. Yin, D. Adam, F. S. Foster, Cancer Res. 2016, 76, 4320-4332.
⁴ J. Weber, P. C. Beard, S. E. Bohndiek, Nat. Publ. Gr. 2016, 13, 639-650.
⁵ K.Valluru, K. Wilson, J. K. Willmann, Radiology. 2016, 280, 332-349.
⁶ S. Y. Emelianov, P.-C. Li, M. O'Donnell, Phys Today 2010, 62, 34-39.
⁷ M. Mehrmohammadi, S. J. Yoon, D. Yeager, S. Y. Emelianov, Curr Mol Imaging 2013, 2, 89-105.
⁸ S. Zackirsson, S. M. W. van de Ven, S. S. Gambhir, Cancer Res 2015, 74, 979-1004.
⁹ K. E. Wilson, S. V. Bachawal, L. Abou-Elkacem, K. Jensen, S. Machtaler, L. Tian, J. K. Willmann, Theranostics 2017, 7, 1463-1476.
¹⁰ K. E. Wilson, S. V Bachawal, J. K. Willmann, Clin. Cancer Res. 2018, clincanres.0417.2018.
¹¹ H. K. Zhang, Y. Chen, J. Kang, A. Lisok, I. Minn, M. G. Pomper, E. M. Boctor, J. Biophotonics 2018, e201800021.
¹² M. Capozza, F. Blasi, G. Valbusa, P. Oliva, C. Cabella, F. Buonsanti, A. Cordaro, L. Pizzuto, A. Maiocchi, L. Poggi, Photoacoustics 2018, 11, 36-45.
¹³ C. Zhang, Y. Zhang, K. Hong, S. Zhu, J. Wan, Nat. Publ. Gr. 2017, 1-9.
¹⁴ A. Vito, H. Alarabi, S. Czorny, O. Beiraghi, J. Kent, N. Janzen, A. R. Genady, S. A. Alkarmi, S. Rathmann, Z. Naperstkow, et al., PLoS One 2016, 11, e0167425.

¹⁵ K. Okumura, K. Yoshida, K. Yoshioka, S. Aki, N. Yoneda, D. Inoue, A. Kitao, T. Ogi, K. Kozaka, T. Minami, et al., Eur. Radiol. Exp. 2018, 2, DOI 10.1186/s41747-018-0036-7.
¹⁶ A. Yazdani, H. Bilton, A. Vito, A. R. Genady, S. M. Rathmann, Z. Ahmad, N. Janzen, S. Czorny, B. M. Zeglis, L. C. Francesconi, et al., J. Med. Chem. 2016, 59, 9381-9389.
¹⁷ R. Rossin, T. Lappchen, S. M. van den Bosch, R. Laforest, M. S. Robillard, J. Nucl. Med. 2013, 54, 1989-1995.
¹⁸ B. M. Zeglis, C. Brand, D. Abdel-Atti, K. E. Carnazza, B. E. Cook, S. Carlin, T. Reiner, J. S. Lewis, Mol. Pharm. 2015, 12, 3575-3587.
¹⁹ H. J. Chung, T. Reiner, G. Budin, C. Min, M. Liong, D. Issadore, H. Lee, R. Weissleder, ACS Nano 2011, 5, 8834-8841.
²⁰ T. S. T. Wang, R. A. Fawwaz, L. Johnson, G. E. Mojdehi, P. M. Johnson, n.d., 21, 767-771.
²¹ K Ogawa, T. Mukai, Y. Inoue, M. Ono, H. Saji, J. Nucl. Med. 2006, 47, 2042-2047.
²² [a] J. Chen, D. S. Hage, 2006, 78, 2672-2683; [b] J. M. Kelly, A. Amor-coarasa, A. Nikolopoulou, W. Till, P. Barelli, D. Kim, C. W. Jr, X. Zheng, C. Bi, B. Hu, et al., 2017, 58, 1442-1450; [c] M. Karimi, S. Bahrami, S. B. Ravari, P. Sahandi, H. Mirshekari, M. Bozorgomid, S. Shahreza, M. Sori, M. R. Hamblin, Expert Opin. Drug Deliv. 2016, 13, 1609-1623; [d] R. Li, H. Yang, D. Jia, Q. Nie, H. Cai, Q. Fan, L. Wan, L. Li, X. Lu, J. Control. Release 2016, 228, 96-106 [e] M. Cristina, H. Struthers, C. Winiger, K. Zhernosekov, R. Schibli, J. Nucl. Med. 2013, 54, 124-132.
²³ Watson, A. D. Binding Studies of Near Infrared Cyanine Dyes with Human Serum Albumin and Poly-L-Lysine Using Optical Spectroscopy Methods By, Georgia State University, 2008.
²⁴ A. Mishra, R. K. Behera, P. K. Behera, B. K. Mishra, G. B. Behera, Chem Rev 2011, 100, 1973-2011.

1. A photoacoustic agent comprising a photoacoustically active moiety coupled to a first bioorthogonal reactive group.

2. The photoacoustic agent of claim 1, wherein the photoacoustically active moiety is selected from a gold nanoparticle, a carbon nanomaterial, a polymer nanoparticle, a genetically encoded chromophore and a near infrared (NIR) dye.

3. The photoacoustic agent of claim 2, wherein the photoacoustically active moiety is a NIR dye.

4. The photoacoustic agent of claim 3, wherein the NIR dye is capable of binding a blood protein.

5. The photoacoustic agent of claim 4, wherein the NIR dye is of Formula (I): 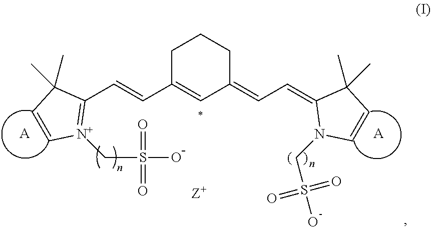
wherein
each is 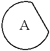
independently aryl;
each n is independently an integer of from 2 to 8;
Z⁺ is a pharmaceutically acceptable countercation; and
* is the site at which the NIR dye is coupled to the first bioorthogonal reactive group.

6. The photoacoustic agent of claim 1, wherein the photoacoustically active moiety is coupled to the first bioorthogonal reactive group by a linker group of the formula: 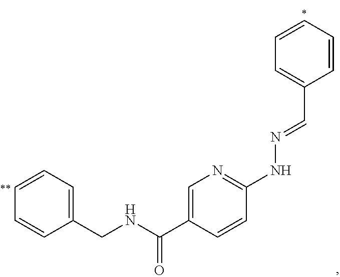 [SMILES: O=C(NCc1ccccc1)c1ccc(N/N=C/c2ccccc2)nc1]
wherein
* is the site that is bonded to the photoacoustically active dye; and
** is the site that is bonded to the first bioorthogonal reactive group.

7. The photoacoustic agent of claim 1, wherein the photoacoustic agent further comprises a targeting entity for a target.

8. The photoacoustic agent of claim 7, wherein the targeting entity is coupled to a second bioorthogonal reactive group that is complementary to the first bioorthogonal reactive group and the first bioorthogonal reactive group and the second bioorthogonal reactive group form a bioorthogonal complex.

9. The photoacoustic agent of claim 8, wherein the first bioorthogonal reactive group and the second bioorthogonal reactive group, in either order, are inverse electron demand Diels Alder reaction pairs, or an azide and a functionalized phosphine, or an azide and a strained alkyne.

10. The photoacoustic agent of claim 9, wherein the first bioorthogonal reactive group and the second bioorthogonal reactive group, in either order, are tetrazine and transcyclooctene.

11. The photoacoustic agent of claim 10, wherein the targeting entity is an antibody, a receptor ligand or a bisphosphonate.

12. The photoacoustic agent of claim 10, wherein the target is a cellular marker for one of inflammation, cancer, a skeletal condition, a heart abnormality, atherosclerosis, angiogenesis and intravascular thrombus formation.

13. The photoacoustic agent of claim 7, of the formula: 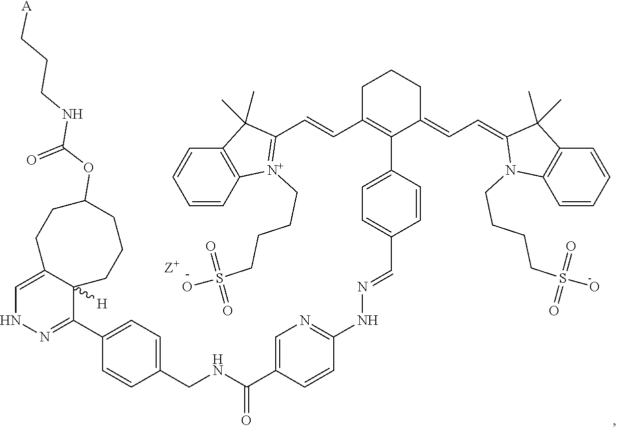
wherein
Z⁺ is the pharmaceutically acceptable countercation; and
A is the targeting entity.

14. A method for detecting a presence of a target in a subject, comprising:
administering a photoacoustic agent comprising a targeting entity for the target as defined in claim 7 to the subject;
allowing the targeting entity to localize to the target; and
imaging the subject using photoacoustic imaging to detect the photoacoustic agent, wherein the detection of the photoacoustic agent indicates the presence of the target in the subject.

15. The method of claim 14, wherein the photoacoustically active moiety is a NIR dye of Formula (I): 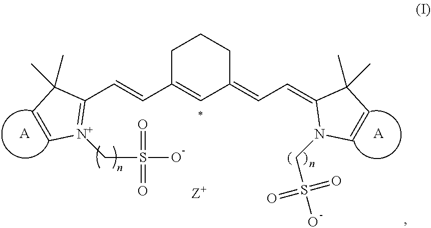
wherein
each is 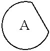
independently aryl;
each n is independently an integer of from 2 to 8;
Z⁺ is a pharmaceutically acceptable countercation; and
* is the site at which the NIR dye is coupled to the first bioorthogonal reactive group.

16. The method of any one of claim 15, wherein the photoacoustically active moiety is coupled to the first bioorthogonal reactive group by a linker group of the formula: 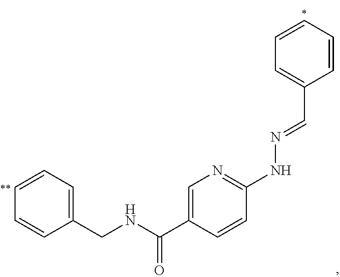 [SMILES: O=C(NCc1ccccc1)c1ccc(N/N=C/c2ccccc2)nc1]
wherein
* is the site that is bonded to the photoacoustically active dye; and
** is the site that is bonded to the first bioorthogonal reactive group.

17. The method of claim 16, wherein the first bioorthogonal reactive group and the second bioorthogonal reactive group, in either order, are inverse electron demand Diels Alder reaction pairs, or an azide and a functionalized phosphine, or an azide and a strained alkyne.

18. A kit comprising:
a photoacoustic agent of claim 1;
a targeting agent, the targeting agent comprising a targeting entity for a target coupled to a second bioorthogonal reactive group that is complementary to the first bioorthogonal reactive group such that the first bioorthogonal reactive group and the second bioorthogonal reactive group form a bioorthogonal complex; and
optionally instructions for use of the targeting agent and the photoacoustic agent in imaging a subject using photoacoustic imaging to detect the photoacoustic agent.

19. The kit of claim 18, wherein the photoacoustically active moiety is a NIR dye of Formula (I): 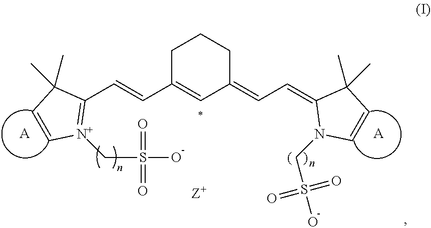
wherein
each is 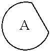
independently aryl;
each n is independently an integer of from 2 to 8;
Z⁺ is a pharmaceutically acceptable countercation; and
* is the site at which the NIR dye is coupled to the first bioorthogonal reactive group.

20. The kit of claim 19, wherein the photoacoustically active moiety is coupled to the first bioorthogonal reactive group by a linker group of the formula: 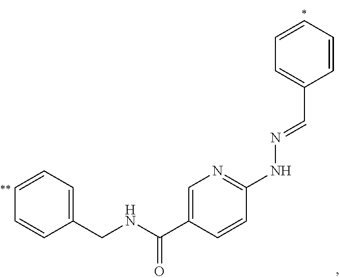 [SMILES: O=C(NCc1ccccc1)c1ccc(N/N=C/c2ccccc2)nc1]
wherein
* is the site that is bonded to the photoacoustically active dye; and
** is the site that is bonded to the first bioorthogonal reactive group.
